(19) United States
(12) Patent Application Publication
(10) Pub. No.: US 2026/0131139 A1
(43) Pub. Date: May 14, 2026

(54) SYSTEM AND METHOD FOR ELECTROPORATION

(71) Applicant: FOCAL MEDICAL TECHNOLOGY LTD, Or Yehuda (IL)

(72) Inventors: Adrian Paz, Petah Tikva (IL); Nadav Ben Daat, Kibbutz Lehavot Haviva (IL); Benny Gamus, Ramat Gan (IL); Avital Spector, Tel Aviv (IL)

(73) Assignee: FOCAL MEDICAL TECHNOLOGY LTD, Or Yehuda (IL)

(21) Appl. No.: 19/445,655

(22) Filed: Jan. 12, 2026

Related U.S. Application Data
(63) Continuation-in-part of application No. PCT/IL2024/050840, filed on Aug. 21, 2024.
(60) Provisional application No. 63/535,072, filed on Aug. 29, 2023.

(51) Int. Cl. A61N 1/05; A61B 8/08; A61B 8/12; A61B 18/00; A61B 18/14; A61N 1/08; A61N 1/32

(52) U.S. Cl. CPC A61N 1/0502; A61B 8/0841; A61B 8/12; A61B 18/1477; A61N 1/08; A61N 1/327; A61B 2018/00547; A61B 2018/00577; A61B 2018/00982; A61N 2001/083

(57) ABSTRACT

A system and method are disclosed to position a 2D array active elements on a 2D array of transperineal stylets in a tissue containing a tumor under guidance by imaging technologies, for evaluating, monitoring, and treating tumors by electroporation.

### RELATED APPLICATIONS

This application is a Continuation in Part of International patent application no. PCT/IL2024/050840 filed on 21 Aug. 2024, which claims the benefit of priority under 35 USC § 119(e) of U.S. Provisional Ser. No. 63/535,072 filed on 29 Aug. 2023, the contents of which are incorporated herein by reference in their entirety.

### FIELD AND BACKGROUND OF THE INVENTION

The present invention, in some embodiments thereof, relates to a system and method for evaluating, monitoring, and treating tumors, more particularly, but not exclusively, by electroporation.

U.S. Pat. No. 8,548,562 appears to disclose, “an imaging and diagnostic system and method to differentiate between malignant and non-malignant tissue of a prostate and surrounding region, wherein the system acquires imaging data from the prostate and surrounding proximal region, and processes the data to differentiate areas of tissue malignancy from non-malignant tissue. A sectioning device or ablative device is provided, wherein the ablative device is operable by automation for receiving the imaging output coordinates and defining the trajectory and quantity of energy or power to be delivered into the malignant tissue. A control system determines calculated energy or power to be deposited into the malignant tissue during ablation, to minimize destruction of the non-malignant tissue within the prostate and surrounding tissue. The system operates on generated ablative device output data.”

Additional background art includes Japanese Utility Application No. 2020520717, U.S. Pat. No. 9,877,788, U.S. Pat. No. 10,575,899, Romanian Application No. 129697 and US patent application No. 2020/086140 and Chinese U.S. Pat. No. 105,616,004, U.S. Pat. No. 8,361,066, US patent application No. 2019/099214, and Collettini, et al., “Image-guided Irreversible Electroporation of Localized Prostate Cancer: Functional and Oncologic Outcomes”, Radiology, 2019, 292:1, 250-257.

Therefore, there is still a need for a system and a method for evaluating tumors and providing reliable focused treatment thereof without causing massive damage to the surrounding tissue.

### SUMMARY OF THE INVENTION

According to an aspect of some embodiments of the invention, there is provided a system for insertion of an array of stylets into tissue, including: a cartridge housing a plurality of stylets each of the stylets including a pointed distal end and a proximal portion connected to the cartridge; and a first template with a plurality of channels through which a distal portion of the stylets pass into the tissue.

According to some embodiments of the invention, the system further includes an actuator configured to move the stylets longitudinally with respect to the first template thereby moving the distal portion of the plurality of stylets longitudinally through the channels of the first template.

According to some embodiments of the invention, the system further includes an x-y actuator for positioning a pusher against a selected stylet of the plurality of stylets and wherein the actuator pushes the selected stylet to the desired depth.

According to some embodiments of the invention, the system further includes a second actuator for detaching the pusher from the selected stylet.

According to some embodiments of the invention, the plurality of stylets are parallel one to the other.

According to some embodiments of the invention, each of the plurality of stylets is oriented longitudinally.

According to some embodiments of the invention, the system further includes a second template, the first template in contact with the tissue and the second template positioned between the first template and the cartridge, wherein the second template is connected to at least a portion of the stylets and wherein the second template is moved longitudinally by the actuator between the first template thereby moving the portion of the stylets longitudinally.

According to some embodiments of the invention, the second template is configured for retracting the stylets for a predetermined distance.

According to some embodiments of the invention, the system further includes a processor running an algorithm configured for the retracting and for positioning active electrodes to a next more superficial depth to be treated.

According to some embodiments of the invention, the retracting is for a distance between ½ cm to 3 cm.

According to some embodiments of the invention, the stylets are configured to be extended and retracted individually through the first template.

According to some embodiments of the invention, the system further includes at least one actuator configured to reversibly extend or retract the stylets to a desired depth within the tissue.

According to some embodiments of the invention, the actuator is configured to move the stylets automatically, or remotely.

According to some embodiments of the invention, the system further includes an imaging modality and wherein the system is configured to be mounted to an imaging modality.

According to some embodiments of the invention, the operator interface is configured for the operator marking on the real time image a region of a procedure.

According to some embodiments of the invention, the marking is performed at various depths.

According to some embodiments of the invention, the system further includes a processor running an algorithm to activate only stylets of the plurality of stylets in the region of a procedure at each of the various depths.

According to some embodiments of the invention, an electronic grid is superimposed on the real time image and wherein the grid markings, coincide with expected positions of the stylets after being pushed into tissue.

According to some embodiments of the invention, the system further includes a processor running an algorithm to activate a remote actuator that will push each stylet of the plurality of stylets into the tissue at the desired depth and within the boundaries of the region of a procedure.

According to some embodiments of the invention, the algorithm in configured to instruct an actuator to push the stylets corresponding to the region of a procedure.

According to some embodiments of the invention, the system further includes a processor running an algorithm to pass the current from a first subset of the stylets to a second subset of the stylets that are intermingled with each other.

According to some embodiments of the invention, the first subset of the stylets are dispersed at equal distance from each other.

According to some embodiments of the invention, the first subset of the stylets and the second subset of the stylets are distributed and intercalated according to a pattern chosen from parallel rows, concentric pattern and other such equidistant patterns.

According to some embodiments of the invention, the pattern is predetermined to facilitate uniformity of current distribution within an area of treatment.

According to some embodiments of the invention, the first subset of the stylets and the second subset of the stylets are is intercalated along a long axis of the shape to be ablated.

According to some embodiments of the invention, the system further includes a bioimpedance module that is connected to the stylets for testing impedance between electrodes on the stylets.

According to some embodiments of the invention, the impedance testing is for differentiating between different types of tissue.

According to some embodiments of the invention, the different types of tissue include benign and malignant tissue.

According to some embodiments of the invention, the impedance testing is for evaluation of a performance and effect on the treatment treated tissue.

According to some embodiments of the invention, the imaging modality is configured to provide imaging by an imaging modality selected from the group consisting of: ultrasound, computed tomography (CT), magnetic resonance imaging (MRI), positron emission tomography (PET) scan, single-photon emission computerized tomography (SPECT) scan, fluoroscopy, endoscopy, laparoscopy, or any combination thereof.

According to some embodiments of the invention, the imaging modality is a Trans Rectal Ultrasound Probe (TRUS).

According to some embodiments of the invention, the imaging modality and the system are configured for independent positioning.

According to some embodiments of the invention, the imaging modality and the system are configured for synchronized positioning.

According to some embodiments of the invention, the system further includes an actuator configured to facilitate synchronized axial movement of the stylets and the imaging modality.

According to some embodiments of the invention, the actuator is configured to facilitate rotation of the imaging modality for imaging stylet positioning.

According to some embodiments of the invention, an angle of rotation is determined by a predetermined position of the stylet in the cartridge relative to the imaging modality.

According to some embodiments of the invention, the imaging modality and the system are configured to be held in pre-determined positions reducing mutual degrees of freedom.

According to some embodiments of the invention, the at least one of the cartridge and the stylet includes an individual actuator for longitudinal movement of an individual stylet.

According to some embodiments of the invention, the individual actuator includes a magnetic tip configured to interface with a magnetic and/or magnetizable means on a tail of an individual stylet of the plurality of stylets for releasably engaging the stylet.

According to some embodiments of the invention, the system further includes a slidable actuator for detaching from the engaged stylet.

According to some embodiments of the invention, each stylet includes a telescopic, removable, and/or retractable inner rod.

According to some embodiments of the invention, each stylet includes a lumen.

According to some embodiments of the invention, the lumen is configured to permit insertion of active electrodes.

According to some embodiments of the invention, each stylet is at least partially covered by a covering including at least one of a sheath and a coating.

According to some embodiments of the invention, the covering is at least one of hydrophobic, hydrophilic, electrically insulating, thermally insulating, and friction reducing.

According to some embodiments of the invention, each of the stylets includes an active electrode of ½ cm to 3 cm length.

According to some embodiments of the invention, the sheath is removable and/or retractable.

According to some embodiments of the invention, each of the stylets is provided with at least two sensing electrodes for bioimpedance measurements.

According to some embodiments of the invention, further including activatable visual markers on the cartridge.

According to some embodiments of the invention, the system where the activable markers are include one or more of LEDs, projected bright dots, and bright project lines.

According to some embodiments of the invention, the activatable markers are produced by a projector situated behind the cartridge.

According to some embodiments of the invention, the system further includes a processor running an algorithm for activating the activatable markings corresponding to the bases of the needles that were chosen to be inserted into tissue for easy identification.

According to some embodiments of the invention, the stylet indicator is a marking on a proximal end or side of the stylet.

According to some embodiments of the invention, the stylet indicator is configured to indicate which stylets are extended and/or at any time.

According to some embodiments of the invention, the stylets are configured for a tissue treatment selected from the group consisting of: irreversible electroporation (IRE), reversible electroporation (RE), instillation of chemotherapeutics, calcium solution or gene therapy, radiofrequency ablation, microwave ablation, laser ablation, photodynamic therapy, and a combination thereof.

According to some embodiments of the invention, the template is configured to provide a variety of densities and patterns of the plurality of stylets.

According to some embodiments of the invention, the plurality of stylets are equally spaced apart.

According to some embodiments of the invention, the plurality of stylets are positioned and spaced in hexagons.

According to some embodiments of the invention, the tissue is a prostate.

According to some embodiments of the invention, the stylets are introduced into the tissue through a perineum.

According to an aspect of some embodiments of the invention, there is provided a method for electro-stimulation of tissue of a subject, the method including: attaching a cartridge with an array of stylets to a cradle of an imaging modality, wherein the cartridge is configured for parallel insertion of stylets into the tissue; introducing the stylets into the tissue to a desired depth, under monitoring by the imaging modality; selecting a desired region of a procedure; executing treatment on the selected region of a procedure; retracting the stylets to a more superficial transverse plane and repeating the previous steps, as needed.

According to some embodiments of the invention, the imaging modality is selected from the group consisting of: ultrasound, computed tomography (CT), magnetic resonance imaging (MRI), positron emission tomography (PET) scan, single-photon emission computerized tomography (SPECT) scan, fluoroscopy, endoscopy, laparoscopy, or any combination thereof.

According to some embodiments of the invention, the imaging modality is a Trans Rectal Ultrasound Probe (TRUS).

According to some embodiments of the invention, the inserting is accomplished manually, automatically, robotically, remotely or any combination thereof.

According to some embodiments of the invention, the retracting is accomplished manually, automatically, robotically, remotely or any combination thereof.

According to some embodiments of the invention, the retracting is in a longitudinal direction and wherein the system is configured to stop periodically for treating a new layer in the transverse and lateral directions.

According to some embodiments of the invention, the region of a procedure is selected manually or automatically.

According to some embodiments of the invention, the region of a procedure is pre-designated.

According to some embodiments of the invention, the treatment is selected from a group including: contact radiofrequency energy, non-contact radiofrequency energy, electroporation, ultrasonic energy, laser energy, gamma radiation, beta radiation, alpha radiation, immunotherapy, or a combination thereof.

According to some embodiments of the invention, the method further includes a preliminary designation step of the region of a procedure.

According to some embodiments of the invention, multiple stylets are configured for acting simultaneously.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIG. 1: A perspective view of a schematic illustration of a system comprising a plurality of electrodes, in accordance with some embodiments of the current invention.

FIG. 2: A cross-section view of a schematic illustration of a system comprising a plurality of electrodes, in accordance with some embodiments of the current invention.

FIG. 3: A schematic illustration of a system comprising a plurality of electrodes with retractable/removable inner stylets/electrodes and retractable/removable sheaths, which may be connected to independent cartridges and/or be inserted and/or retracted independently, in accordance with some embodiments of the current invention.

FIG. 4: A schematic illustration of a system comprising a plurality of electrodes combined with a TRUS probe comprising a plurality of electrodes, in accordance with some embodiments of the current invention.

FIG. 5: A schematic illustration of positioning of a system with an integrated TRUS probe, in accordance with some embodiments of the current invention.

FIG. 6: A cross-sectional schematic illustration of positioning of a plurality of stylets ensuring equal spacing between any consecutive stylets and plurality of stylets connected simultaneously to positive and negative polarities of the electrical source in rows, in accordance with some embodiments of the current invention.

FIG. 7: A cross-sectional schematic illustration of positioning of a plurality of stylets connected simultaneously to positive and negative polarities of the electrical source, in accordance with some embodiments of the current invention.

FIG. 8: A cross-sectional schematic illustration of positioning of a plurality of stylets wherein an area of ablation is selected, in accordance with some embodiments of the current invention.

FIG. 9: An exemplary ultrasound image of a plurality of stylets wherein an area of ablation is selected, in accordance with some embodiments of the current invention.

FIG. 10: A block diagram of evaluation of an electroporation system, in accordance with an embodiment of the current invention.

FIG. 11: A flow chart of treatment and/or mapping of a tumor using an electroporation system, in accordance with an embodiment of the current invention.

FIGS. 12A and 12B: Flow charts of treatment and/or mapping of tissue, in accordance with embodiments of the current invention.

FIGS. 13A and 13B: A perspective view and side view, respectively, of schematic illustrations of positioning of a system with an integrated TRUS probe, in accordance with some embodiments of the current invention.

FIGS. 14A and 14B: A perspective view and side view, respectively, of schematic illustrations of an exemplary transperineal stylet, in accordance with some embodiments of the current invention.

FIG. 15 is a flow chart illustration of a method of executing a procedure in accordance with an embodiment of the current invention.

FIG. 16 is a block diagram illustrating a system for inserting stylets into tissue in accordance with an embodiment of the current invention.

### DESCRIPTION OF SPECIFIC EMBODIMENTS

The present invention, in some embodiments thereof, relates to a system and method for evaluating, monitoring, and treating tumors, more particularly, but not exclusively, by electroporation.

### Overview

The present invention, in some embodiments thereof, relates to a system and method for evaluating, and/or monitoring, and/or treating tumors, more particularly, but not exclusively, by electroporation.

Some embodiments relate to a system and/or method to treat and/or map tissue using a two-dimensional (2D) and/or quasi-two-dimensional array of stylets which may be moved through the tissue layer by layer. For example, the system may be used to map and/or treat tissue which may contain one or more cancerous tumors. Optionally, the plurality of stylets may be inserted into a volume of tissue trans-perennially. Optionally, the tissue may be, part of, or the entire prostate gland.

According to some embodiments, the system may be configured to position a 2D array of active elements (preferentially electrodes) on a 2D array of transperineal stylets (e.g., probes, needles) in the prostrate. Optionally, each transperineal stylet may include multiple active elements making it a three-dimensional (3D) array of active elements. Optionally, the system may be configured to function under guidance. Optionally, the guidance may include a TRUS (Trans Rectal Ultrasound Probe), and/or other imaging technologies.

According to some embodiments, the system and/or method may be similar to current transrectal ultrasound (TRUS) prostrate biopsies (e.g., transperineal template-guided prostate mapping biopsy (TTMB)) and/or Brachytherapy, but may advantageously include a large number of electrodes and/or stylets which may be moved together as a group and may be inserted at once thereby making the procedure much faster and less damaging.

Additionally, or alternatively, the method and/or the system may be used for various forms of treatment (e.g., ablation, radiofrequency techniques, etc.) and/or various types of tomography (e.g., impedance tomography [e.g., bioimpedance]) which may be performed serially in 2D layers (e.g., different stylets may be active for various 2D patterns of treatment including different non-continuous regions). According to some embodiments, the disease requiring treatment may be a tumor. For example: the tissue may be some or the entire prostate gland, or other tissue tumor. The system may more accurately discern between tumoral tissue and normal tissue especially determining the boundaries and multifocal foci.

In some embodiments, a module will be connected to the stylets for measuring bioimpedance. For example, the bioimpedance module may include an impedance analyzer. For example, the impedance analyzer may generate the alternating current, measure the voltage, and/or calculates the impedance. It can be a standalone device or integrated into other systems. Optionally electrodes on the stylets are used to make contact with the tissue. Optionally, the bioimpedance module may include signal conditioning circuitry. For example, the signal conditioning circuitry may be used to amplify and/or filter measured signals. Conditionally may improve accuracy of measurements and/or reduce noise. Optionally, the bioimpedance module will include a data acquisition system. For example, the data acquisition system may be used to collect and/or store impedance data for analysis. Optionally, the bioimpedance module will include a processor and/or analysis software. For example, collected data may be analyzed using specialized software to extract meaningful information about the tissue.

According to some embodiments, the system may include a device for parallel insertion of stylets into tissue. Optionally, the stylets may be thin (e.g., For example, the system may include a plurality of stylets, connected to a cartridge, inserted through a template (e.g., a front plate through tight channels), which may act as a guide for insertion of the stylets into tissue. According to some embodiments, the cartridge may slide with respect to the template. According to some embodiments, the stylets may be releasable, and/or may be disconnected from the cartridge.

According to some embodiments, an actuator may be provided for remote and/or manual and/or automatic translation of the cartridge towards and/or away from the template. According to some embodiments, the actuator may be connected to a controller. In some embodiment a sensor (e.g., a Transrectal Ultrasound [TRUS] Probe may move with [e.g., be retracted along with] the stylets). Alternatively, or additionally, the stylets and the sensor may move independently.

According to some embodiments, the guiding means (template) may permit parallel translation of the cartridge in relation to the template. Optionally, the guiding and/or sliding means may facilitate passage of the stylets. Optionally, the stylets may pass therethrough and/or be guided through corresponding parallel channels in the front-plate. Optionally, the guiding means may facilitate parallel insertion of the stylets. Optionally, the guiding means may be parallel rods with and/or without linear bearing and/or bushing. Optionally, this may provide smooth controlled parallel translation of the cartridge towards the template and/or away from it. Optionally, the guiding means may be a linear rail guide.

According to some embodiments, the array may include a plurality of stylets. Optionally, the plurality of stylets may be arranged in a two-dimensional (2D) array. Alternatively, or additional, the plurality of stylets may be arranged in a quasi-two-dimensional array, e.g., the stylets (e.g., 0.01 mm to 2 mm thick). Optionally, the distal ends of the stylets and/or electrodes associated with the stylets may not all be planar. Optionally, the stylets may be staggered within a range of longitudinal distances, etc.

According to some embodiments, stylets of different polarities may be interspaced and/or may create a pattern selected from the group of: interdigitating hexagons, interdigitating polygons, concentric polygons, alternating straight or curved lines, interdigitating spirals, etc. or any combination thereof. According to some embodiments, the system and/or method may include a control modality and a display modality for selecting which stylets may be electrically connected to the system and which may be disconnected.

In some embodiments, the electrically active portion of the stylet (e.g., the portion of the stylet in electrical contact with the tissue and/or an electrode) may have a finite length. Optionally, the electrically active portion of the stylet may have a length ranging between about 0.01 mm to about 0.1 mm, and/or between about 0.1 mm to about 0.5 mm, and/or between about 0.5 mm to about 1.5 mm, and/or between about 1.5 mm to about 5 mm, and/or between about 5 mm to about 10 mm, and/or between about 10 mm to about 20 mm.

According to some embodiments, the longitudinal distance electrical portions of different stylets may range between about 0.1 mm to about 0.5 mm, and/or between about 0.5 mm to about 1 mm, and/or between about 1 mm to about 5 mm, and/or between about 5 mm to about 10 mm, and/or between about 10 mm to about 20 mm.

According to some embodiments, the stylet may be moved longitudinally through the tissue. Optionally, the stylets may be stopped periodically within the tissue and/or activated to treat a horizontal layer of tissue (e.g., a layer of tissue parallel to the transverse plane of the stylets). Optionally, the longitudinal distance between stops and/or layers may be between about 1 mm to about 3 mm, and/or between about 3 mm to about 10 mm, and/or between about 10 mm to about 20 mm.

According to some embodiments, the stylets may be inserted together in an array. Optionally, inserting stylets together may facilitate the stylets stylet remaining in fixed relative alignment as they are inserted. Optionally, each stylet may be extended and/or retracted individually. Optionally, each stylet may be extended and/or retracted manually and/or automatically.

According to some embodiments, the stylets may be marked. Optionally, each stylet may be marked on the proximal side (e.g., with numbers and/or LED's). Optionally, each stylet may may be marked such that a user may see which stylets are extended and/or activated (e.g., the electrode is actively receiving current) at any time.

According to some embodiments, the array may include about 2-10 layers, and/or 3-15 layers, and/or 1-20 layers. Optionally, the array may include a matrix of stylets. For example, the matrix may include between 2 to 4 stylets, and/or between 4 to 10 stylets, and/or between 10 to 20 stylets, and/or between 20 to 50 stylets across in a lateral direction and/or in a transvers direction. Optionally, the number of stylets in the lateral direction may be different from the number of stylets in the transvers direction. Optionally, the number of stylets in the lateral direction may be the same as the number of stylets in the transvers direction. In some embodiments, more than one array of stylets may be provided and/or may move independently. Optionally, multiple arrays of stylets may be interlaced. Optionally, an array of stylets may be pre-packaged.

According to some embodiments, the stylets may be equally spaced. Alternatively, or additionally, the stylets may be spaced differentially (for example, the stylets may be spaced densely in one area and less densely elsewhere. Optionally, the number of stylets may be denser towards the center of the array than at the periphery. Optionally, the number of stylets may be denser towards the periphery of the array than at the center. Optionally, the number of stylets may be denser on one side of the array than on another. Optionally, the density of the stylets may vary, e.g., denser in some regions than in others. Optionally, spacing between any two consecutive stylets may be equal. Optionally, spacing between any two consecutive stylets may vary. In some embodiments, stylets of opposing polarity may be distanced at a constant gap while stylets of similar polarity may be distributed in a different gap and/or in varying gaps.

According to some embodiments, the array may be circular, oval, square, rectangular and/or another shape and/or irregularly shaped. For example, the shape of the array of stylets may be customized according to the shape of the region being tested and/or treated. Optionally, the stylets may be positioned and/or spaced in triangles, squares, pentagons, hexagons, and/or circles.

According to some embodiments, the lateral distance between any two adjacent stylets may range between 0.1 to 0.5 mm and/or between 0.5 to 3 mm and/or between 3 to 10 mm and/or between 10 to 20 mm, and/or between about 3 mm to about 15 mm. Preferably, the lateral distance between any two adjacent stylets may be about 5 mm.

According to some embodiments, the stylets may have a thickness ranging between 0.01 mm to 1 mm, and/or between 0.2 mm to 0.7 mm, and/or between 1 to 2 mm. Preferably, the thickness of the stylets may be less than 0.5 mm. According to some embodiments, the use of fine stylets may cause less tissue trauma. Optionally there may be different types and/or sizes of stylets with one array and/or within a set of interlaced arrays.

According to some embodiments, each stylet may be at least partially covered by a sheath. Optionally, the sheath may be electrically insulating, thermally insulating, or both. Optionally, the sheath may be composed of a polyimide tube. Optionally, the sheath may be removable and/or retractable.

According to some embodiments, each stylet may comprise a telescopic removable and/or retractable inner rod. Optionally, the sheaths and/or the rods may be connected to a template. Optionally, the template may be parallelly translatable in relation to a cartridge to which the stylets may be attached. Optionally, a cartridge may be configured to facilitate retraction and/or removal of the sheath and/or the rods relative to the stylets.

According to some embodiments, the stylets may be hollow and/or may include a lumen. Optionally, the hollow stylets may facilitate insertion of one or more active electrodes through them. Optionally, the lumen of the stylets may include electrodes, and/or rods, and/or wires.

According to some embodiments, the stylets may be electroconductive. Optionally, the stylets may be composed, at least in part, from an electroconductive material, such as stainless steel, copper, etc. Optionally, the stylets may be composed, at least in part, from an electroconductive material capable of conducting at least about 10 V, and/or at least about 50 V, and/or at least about 100 V, and/or at least about 200 V, and/or at least about 300 V. Optionally, the electroconductive stylets may be wired to a multi-pin electrical connector.

According to some embodiments, the stylets may be electrically connected to an electrical circuit, e.g., a printed circuit board (PCB). Optionally, the electrical circuit may include a controller. Optionally, the controller may be installed on the baseplate. Optionally, the system may be mounted on an existing brachytherapy platform.

According to some embodiments, the controller may include individual on/off switches (relays) for some or all of the stylets. Optionally, the controller may control and/or sense the position of the stylets and/or a sensor (e.g., a TRUS sensor). For example, the controller may synchronize movement and/or activation and/or reading of the sensor and/or the stylets. Optionally, the controller may include a predetermined program of transverse stylet locations and/or predetermined pattern of longitudinal stylet stop locations and/or a predetermined timing of stylet activation. For example, the controller may move and/or activate the stylets according to the predetermined program. According to some embodiments, the controller may be automated, and/or manually controlled, and/or remotely controlled.

According to some embodiments, the system may include one or more stepper motors. Optionally, the stepper motors may be configured for insertion and/or retraction of a 2D array and/or imaging sensor. Optionally, two or more stepper motors may be configured for insertion of multiple transperineal probes simultaneously and/or consecutively. Optionally, two or more stepper motors may be configured for individual and/or group control of the positioning and or extension of the stylets and/or array. Optionally, two or more stepper motors may be configured for controlled insertion of a 2D array of transperineal stylets. Optionally, two or more stepper motors may be configured for synchronization of positioning of the transperineal stylets and/or an imaging sensor, e.g., a TRUS imaging probe.

According to some embodiments, since the stylets are thin and connected in an array, multiple stops may be made and precise control over the affected area may be affected. For example, the stylets may be retracted in the longitudinal direction, such that the system may stop every few millimeters to treat a new layer in the transverse and/or lateral directions.

According to some embodiments, the stylets may be used in the treatment of tumors. Optionally, the stylets may be used in irreversible electroporation (IRE), reversible electroporation (RE), instillation of chemotherapeutics, calcium solution or gene therapy, radiofrequency ablation, microwave ablation, laser ablation, photodynamic therapy, and/or a combination thereof.

According to some embodiments, the controller may be programmed to deliver a therapeutic effect e.g., as a result of focused ablation, electroporation, electrochemical or chemo-electroporation between local pairs of electrodes. Optionally, the controller may be programmed to deliver the therapeutic effect to the locations with high risk of disease. Optionally, the controller may be programmed to deliver the therapeutic effect interstitially at the location of the interstitial stylets. Optionally, the stylets may include catheters provided with longitudinal channels, and/or tubes. Optionally, the treatment modality may be delivered through the stylet lumen.

According to some embodiments, the stylet array with and/or without the back and/or template may be disposable, e.g., a disposable cartridge. Optionally, the system may include various multi-use components which may be utilized in conjunction with new sterile and/or separately sterilizable disposable stylets. Optionally, various elements of the device, such as the controller and/or the rail, etc., may be stationary and/or may be used multiple times.

According to some embodiments, the controller may be programmed to deliver the therapeutic effect selected from a group comprising: contact radiofrequency energy, non-contact radiofrequency energy, electroporation, ultrasonic energy, laser energy, gamma radiation, beta radiation, alpha radiation, immunotherapy, or a combination thereof.

According to some embodiments, the controller may be programmed to deliver current at a plurality of frequencies between various groups of said plurality of electrodes. Optionally, the current may be non-ablative electrical current. Optionally, the current may be ablative electrical current. Optionally, a plurality of frequencies may pass between various groups of said plurality of electrodes. Optionally, the plurality of frequencies may pass between various groups of said plurality of electrodes sequentially and/or simultaneously. Optionally, the plurality of frequencies may pass between various groups of said plurality of electrodes in pulses. Optionally, the reaction of the tissue to energy may be used for diagnostic purposes. Optionally, the reaction of the tissue to energy may be used for treatment.

According to some embodiments, the controller may be configured to determine the location of at least 2 stylets. Optionally, the controller may be configured to control positioning of the plurality of stylets, plurality of electrodes, or both. Optionally, the plurality of stylets may be introduced into the volume of tissue in approximately parallel directions.

According to some embodiments, the controller may be programmed to calculate a characteristic of the interaction between the signal and the tissue at the plurality of frequencies and at a plurality of locations. Optionally, the characteristic may be an impedance characteristic.

According to some embodiments, the controller may be programmed to detect and/or calculate impedance characteristics at one or the plurality of frequencies at a plurality of locations. According to some embodiments, the controller may be programmed to perform a comparison between the found impedance characteristics at each location and reference characteristics of non-diseased and diseased tissue. According to some embodiments, the controller may be programmed to determine the risk of disease at each particular location from the plurality of locations, within the volume of tissue.

According to some embodiments, the controller may be programmed to deliver ablative energy such as: radiofrequency current or electroporation. According to some embodiments, the controller may be programmed to deliver ablative energy in bipolar or multipolar mode between 2 or more of the electrodes to ablate tissue at locations showing high risk of disease.

According to some embodiments, the impedance characteristic may be selected from a group of: impedance magnitude, impedance phase, impedance phase angle, etc. According to some embodiments, the risk of disease may be based on calculating a percentage difference between the detected impedance characteristic value at each location and reference values. According to some embodiments, a comparison may be performed between the detected characteristic at each location and reference characteristics of non-diseased and diseased tissue.

According to some embodiments, a map such as a tri-dimensional (3D) map, may be generated of the tissue volume. For example, the map may depict the risk of disease at each location and/or an electrical characteristic at each location and/or a reaction to an electrical signal at each location and/or an estimated exposure to a signal (e.g., an ablative signal and/or a therapeutic signal and/or an exploratory signal) at each location and/or or a confidence level of any of the above. Alternatively, or additionally, a signal may be transmitted from the stylets while they are moving through the tissue.

According to some embodiments, the map of risk of disease may be fused with an imaging map of the tissue using information from additional imaging modalities. Optionally, combining techniques may provide a more accurate histological map of the tissue. Optionally, the additional imaging modalities may be selected from the group including: ultrasound, computed tomography (CT), magnetic resonance imaging (MRI), positron emission tomography (PET) scan, single-photon emission computerized tomography (SPECT) scan, fluoroscopy, endoscopy, laparoscopy, or any combination or fusion of modalities.

According to some embodiments, the impedance map may be fused with previous imaging map of ultrasound, computed tomography (CT), Magnetic resonance imaging (MRI), positron emission tomography (PET) scan, single-photon emission computerized tomography (SPECT) scan, fluoroscopy, endoscopy, laparoscopy, or any combination or fusion of modalities.

According to some embodiments, the impedance map may be fused with concomitant imaging map of ultrasound, computed tomography (CT), Magnetic resonance imaging (MRI), positron emission tomography (PET) scan, single-photon emission computerized tomography (SPECT) scan, fluoroscopy, endoscopy, laparoscopy, or any combination or fusion of modalities. Optionally, mapping non-ablative electrical current may be delivered simultaneously, concomitantly or sequentially, to treatment in order to monitor the progress of the treatment.

According to some embodiments, the risk of disease of each voxel may be calculated based on the impedance data and on from previous and/or concomitant imaging modalities such as: ultrasound, computed tomography (CT), Magnetic resonance imaging (MRI), positron emission tomography (PET) scan, single-photon emission computerized tomography (SPECT) scan, fluoroscopy, endoscopy, laparoscopy, or any combination or fusion of modalities.

According to some embodiments, an additional interstitial imaging modality such as optical coherence tomography, may be used in conjunction with impedance data to calculate the risk of disease of some or all the voxels.

According to some embodiments, the risk of disease of each voxel from these multiple data sets may be assigned using advanced algorithms such as neurol networks and deep learning. Optionally, training of such AI algorithms may be using them in comparation with detailed 2D and/or 3D tumor or disease histological mapping of tissue. Optionally, such algorithms may improve continuously with additional data.

According to some embodiments, additional patient clinical, serologic and genetic and proteomic data may be used to improve accuracy.

According to some embodiments, the system may be used to evaluate tumors within a tissue.

According to some embodiments, the system for evaluating tumors within a tissue may include a 2D array of electrodes which may be introduced into the tissue (e.g., prostate) and used for 2D electrical tomography. Optionally, the electrodes may be inserted into the tissue in a plurality of locations. Optionally, the electrodes may be located on a plurality of stylets. Optionally, each or the plurality of stylets may have a plurality of electrodes. Optionally, the electrodes may use an impedance characteristic. Optionally, the impedance characteristic may provide 2D and/or 3D tomography. Optionally, various currents (e.g., various spectroscopic techniques) may be used. Optionally, the current may be non-destructive. Optionally, the non-destructive (e.g., non-ablative) current is delivered between 2 or more electrodes, adjacent one of the plurality of locations. Optionally, non-destructive electroporation may be used to test sensitivity and/or recovery of the tissue. Optionally, the spectroscopic and impedance data may be combined. Optionally, the combined data may be used to provide a 2D and/or 3D histological map of the tissue.

According to some embodiments, (for example where tumor tissue is more sensitive to electroporation than normal tissue), monitoring the change in tissue impedance with initiation of electroporation may assist in refining the tumor risk at each voxel. Optionally, after an initial mapping of the tissue impedance at each voxel, electroporation may be initiated between all, and/or some, pairs of nearby electrodes and the change in impedance detected and this data used to refine the determination of tumor risk at each voxel.

According to some embodiments, a method of diagnosis may include positioning a plurality of electrodes in tissue of interest, passing a current between different groups of said plurality of electrodes, recording an effect of location on said electrical signals, and mapping a property of said tissue based on said effect of location on said electrical signal. Optionally, the current may be alternating and/or direct current.

According to some embodiments, a plurality of currents may be delivered. Optionally, the plurality of currents may be delivered, sequentially, simultaneously, concomitantly, in pulses, and/or a combination thereof. Optionally, the plurality of currents may be delivered concomitantly using a complex wave pattern.

According to some embodiments, the current may be non-ablative. Optionally, the non-ablative current may be delivered sequentially, simultaneously and/or in pulses. Optionally, the non-ablative current may be delivered each time between at least 2 electrodes. Optionally, the non-ablative current may be delivered adjacent to one of a plurality of high-risk locations.

According to some embodiments, the current may be ablative. Optionally, the ablative current may be delivered sequentially, simultaneously and/or in pulses. Optionally, the ablative current may be delivered each time between at least 2 electrodes. Optionally, the ablative current may be delivered adjacent to one of a plurality of high-risk locations. Optionally, the ablative energy may be delivered such that the current paths of the pulses may intersect at a particular location.

According to some embodiments, the plurality of electrodes may be mounted on a plurality of stylets. Optionally, the stylets may be inserted through undamaged tissue until said electrodes reach the target tissue (e.g., prostate tissue). Optionally, the one or more of the plurality of stylets may be inserted trans-perennially.

According to some embodiments, recording the effect of location on the electrical signals may include a time dependent change in said property resulting from said passing alternating current. Optionally, the recording may include an impedance.

According to some embodiments, a system is described comprising a plurality of electrodes, a plurality of interstitial stylets configured for positioning the plurality of electrodes within a volume of tissue, wherein each of the plurality of stylets may be provided with at least one of said plurality of electrodes and at least one of said of plurality of stylets may include a plurality of the electrodes, and a controller in communication with the stylets.

According to some embodiments, the controller may be programmed to deliver energy in between groups of the plurality of electrodes to ablate tissue at locations showing high risk of disease, wherein the current paths are intersecting at a particular location causing a therapeutic effect at their intersection and having lesser effect at the contact of the electrodes with the tissue. Optionally, the controller may be further programmed to deliver energy between groups of the plurality of electrodes simultaneously, sequentially, in pulses, and/or a combination thereof.

According to some embodiments, the therapeutic, or ablative electrical energy may be delivered in sequential pulses between 2 or more electrodes and/or groups of electrodes. Optionally, the current paths of the pulses may intersect at a particular location. Optionally, the pulses may have a duty cycle of between about ½ to about 1/25, or between about ⅓ to ⅕. Optionally, heating and injury to the tissue adjacent to the electrodes surface, may be prevented and the energy may be delivered evenly in the volume of tissue preventing hot or cold spots. In some embodiments, cold ablation is achieved. For example, low energy electrical signal through nearby electrodes may cause tissue ablation without heat. For example, the electrical signal may increase the sensitivity to cells to destruction by another means. For example, in electroporation electrical pulses may create temporary pores in cell membranes. The pores may allow molecules, such as DNA, RNA, or drugs, to be introduced into the cell and/or ablate the cell. Optionally, a signal may be transmitted through the tissue from a stylet to an electrode outside the target tissue (e.g., monopolar).

According to some embodiments, the system described herein allows precise location of a tumor to be measured, and/or treated. Optionally, measurement and treatment may be carried out using the same electrodes. Optionally, measurement and treatment may be carried out without moving the electrodes. Optionally, measurement may be carried out by the electrodes which may be repositioned prior to treatment for increased effect. Optionally, during treatment (e.g., either while ablating and/or during breaks in treatment) the progress of the treatment may be evaluated by one or more of the measurement techniques described above. Optionally, this would allow more reliable positioning of the electrodes and/or reposition of the electrodes. Optionally, this may allow more accurate treatment, as electrode positions and current may be adjusted during treatment for maximum effect. Optionally, frequency, duty cycle, intensity, electrode position, current intersection, pulse time and duration, number of electrodes, etc. and/or a combination thereof may be adjusted.

According to some embodiments, a method for treatment is described comprising a plurality of electrodes in a tissue of interest, a plurality of interstitial stylets configured for positioning said plurality of electrodes within a volume of tissue, passing alternating electric current between different groups of said plurality of electrodes, and triggering destruction of diseased tissue by said passing, thereby providing a therapeutic effect.

According to some embodiments, the passing may be selected from the group including: focused ablation, electroporation, electrochemical or chemo-electroporation between local pairs of electrodes, and/or local groups of electrodes.

According to some embodiments, the therapeutic effect may be delivered to locations with elevated risk of disease. Optionally, the therapeutic effect may be delivered interstitially at the location of the interstitial stylets. Optionally, the therapeutic effect may be selected from a group including: contact radiofrequency energy, non-contact radiofrequency energy, electroporation, ultrasonic energy, laser energy, gamma radiation, beta radiation, alpha radiation, immunotherapy, and/or a combination thereof.

According to some embodiments, electrical ablation may induce electroporation. Optionally, electroporation may be delivered in pulses. Optionally, electroporation may be used in conjunction with one or more chemotherapeutic compounds. Optionally, the electroporation used in conjunction with one or more chemotherapeutic compounds may be synergistic. Optionally, the chemotherapeutic may be an anti-cancer compound. Optionally, the anti-cancer compound may be a small molecule, a biological molecule such as an antibody, a metal, an organometallic compound and/or a radio isotope.

According to some embodiments, electrical ablation may be delivered continuously and/or in pulses. Optionally, electrical ablation may induce tissue heating. Optionally, electrical ablation in pulses may reduce the damage to the surrounding, non-diseased tissue. Optionally, the destruction of tissue may be less than about 0.5/5, less than about ⅕, less than about ⅖, or less than about ⅗ of said volume of tissue.

According to some embodiments, the method may include passing alternating electric current between different groups of said plurality of electrodes, recording an effect of location on said electrical signals, and mapping a property of said tissue based on said effect of location on said electrical signal. Optionally, mapping may be performed before, during and/or after the treatment. Optionally, the treatment may be adjusted during the procedure based on the mapping.

According to some embodiments, the system and/or method may be controlled by a control unit. Optionally, the controller may be automated. Optionally, evaluation of the tumor, e.g., position, histological mapping, etc. may be automated. Optionally, a machine learning algorithm may be used to determine high risk tissue. Optionally, treatment of the tumor may be automated e.g., the treatment selected, the optimal position of a plurality of stylets, the optimal position of a plurality of electrodes, the current to be used, the pulse duration and or rate, duty cycle, chemotherapeutic, etc. Optionally, the automated system may control variation of current delivered, sequential, simultaneous, concomitant, in pulsed, and/or a combination thereof. Optionally, the plurality of currents may be delivered concomitantly using a complex wave pattern between pairs of electrodes and/or groups of electrodes.

According to some embodiments, the system and/or method may include connecting multiple stylets to an electrical generator source, connecting stylets to opposite electrical polarities, wherein some of the stylets are connected to the positive polarity and some of the stylets are connected to the negative polarity, applying the electrical current to multiple stylets connected to the positive polarity and to the negative polarity at the same time. Alternatively, or additionally, all of the stylet may have the same polarity and/or signals may be passed to an electrode external to the region of a procedure and/or stylet and/or device.

According to some embodiments, the system may include a graphic interface. Optionally, the graphic interface may include a display including data from one or more imaging processes, such as ultrasound, MRI, CT, X-ray, impedance tomography, etc. Optionally, data from one or more imaging processes may be merged or fused. Optionally, one or more stylets may be observed on the graphical display. Optionally, one or more stylets may be selected from the graphical display. Optionally, one or more stylets may be activated from the graphical display and/or selected for activation, e.g., the polarity, frequency, duty cycle, voltage, current, etc.

According to some embodiments, the type of treatment applied may be selected from the graphical display, e.g., irreversible electroporation (IRE), reversible electroporation (RE), instillation of chemotherapeutics, calcium solution or gene therapy, Radiofrequency ablation, microwave ablation, laser ablation, photodynamic therapy and/or a combination thereof.

According to some embodiments, robotic feedback may control the stylet retraction and/or switching of the active stylet/s according to the desired regions of interest at each insertion depth.

Some embodiments relate to a method for treating prostate cancer comprising:
introducing trans-rectal ultrasound (TRUS) in the rectum for imaging while connected to a dedicated cradle (urological stepper),
attaching the device to a cradle, wherein the device is configured for parallel insertion of thin stylets into tissue, comprising of: multiple stylets, connected to a cartridge, inserted into a template through tight channels, acting as a guide for insertion of the stylets into tissue, to the cradle, with the template firmly attached to and/or pushed up against the perineum,
introducing the stylets into the perineum and into the prostate to the desired depth, under TRUS monitoring, automatically or manually,
selecting the desired region of a procedure, for example using a graphical interface,
performing electroporation on the region of interest, with multiple stylets acting simultaneously, and
automatically or manually retracting the stylets to a more superficial transverse plane and repeating the procedure, as needed.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

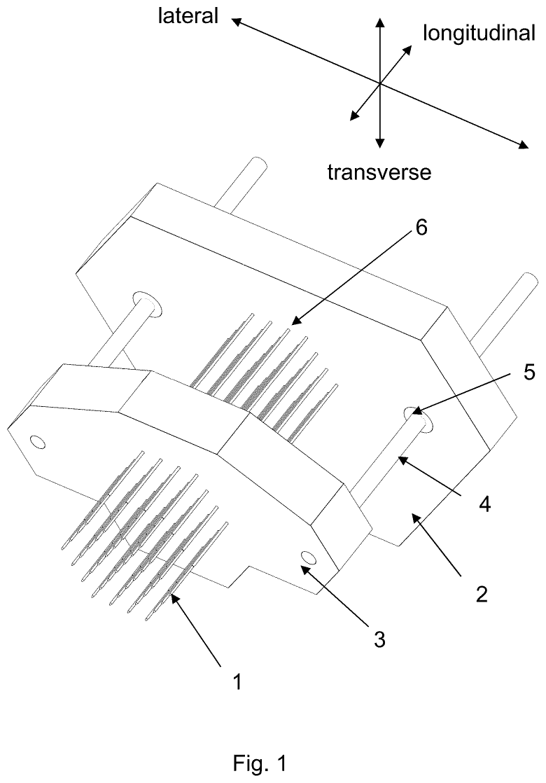

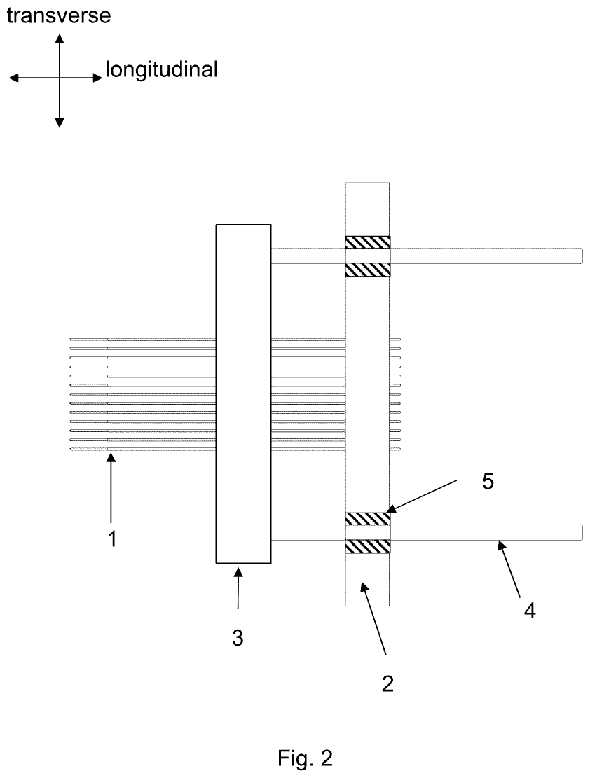

FIG. 1 is a perspective view of a schematic illustration of a system comprising a plurality of electrodes in accordance with some embodiments of the current invention. FIG. 2 is a cross-section view of a schematic illustration of a system comprising a plurality of electrodes in accordance with some embodiments of the current invention. For example, the system may include a plurality of stylets 1. Optionally, a proximal portion of each stylet 1 is connected to a cartridge 2. Optionally, stylets 1 may be connected with their distal ends and/or an active component (e.g., an electrode) coplanar. Alternatively, or additionally, the distal ends of stylets 1 may be offset longitudinally (e.g., forming a 3D array) with their distal ends and/or an active component (e.g., an electrode) not coplanar. In some embodiments, stylets 1 pass through a template 3 and/or channels 6. Optionally, cartridge 2 may move and/or slide with respect to the template 3. For example, sliding of cartridge 2 may cause synchronized and/or simultaneous and/or longitudinal movement of stylets 1. Optionally, movement of the cartridge 2 is guided with one or more parallel guidance rods 4 and linear bearing and/or bushing 5.

According to some embodiments, the system may include a device for parallel insertion of thin stylets into tissue. Optionally, a plurality of stylets 1 are connected to cartridge 2. In some embodiments, stylets 1 may pass through template 3. For example, stylets may pass through tight channels 6 in template 3. Optionally, channels 6 may act as a guide for insertion of stylets 1 into tissue. According to some embodiments, the cartridge 2 may be slidable. According to some embodiments, the stylets 1 may be releasable, and/or may be disconnected from the cartridge.

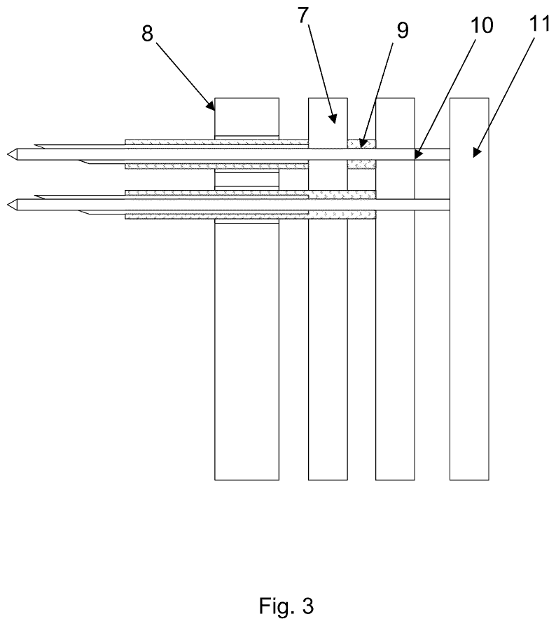

FIG. 3 is a schematic illustration of a system comprising a plurality of electrodes in accordance with some embodiments of the current invention. For example, stylet array 7, is inserted via a guide matrix 8, with retractable sheath 9 and telescopic removable and/or retractable inner stylet 10.

According to some embodiments, each stylet 10 may be at least partially covered by a sheath 9 and/or a coating. Optionally, sheath 9 may be electrically insulating, thermally insulating, or both. Optionally, sheath 9 may be composed of a polyimide tube. Optionally, sheath 9 may be removable and/or retractable. Alternatively or additionally, a coating may be fixed to the stylet. For example, the stylet may include an active electrode zone (e.g., exposed from the sheath and/or coating) of length between 1 mm to 5 mm and/or between 5 mm to 30 mm length. Optionally, a coating and/or sheath may include a friction reducing layer. For example, the friction reducing coating and/or sheath may be hydrophobic coating and/or hydrophilic. Optionally a coating and/or a sheath may include silicone, a Fluoropolymers (e.g., PTFE (Teflon) and/or FEP (fluorinated ethylene propylene)) and/or a lubricant.

According to some embodiments, each stylet 10 may comprise a telescopic removable and/or retractable inner rod. Optionally, the sheaths 9 and/or the stylets 10 may be connected to a cartridge (not shown) that is parallelly translatable in relation to the plate 11 to which the stylets 10 may be attached.

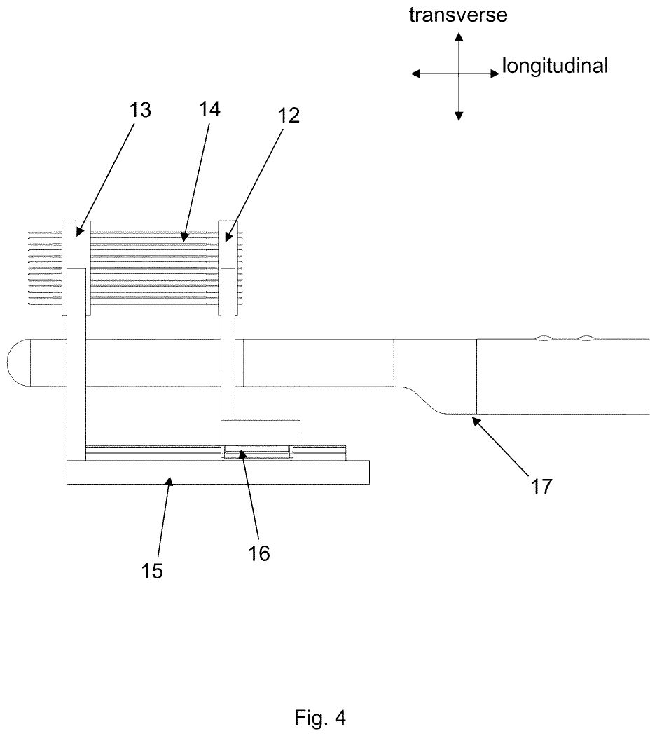

FIG. 4 is a schematic illustration of system comprising a plurality of electrodes combined with a TRUS probe comprising a plurality of electrodes in accordance with some embodiments of the current invention. For example, a system including cartridge 12 with multiple stylets 14 and template 13, installed into a customized urological stepper (cradle) 15 with linear rail 16 and combined with TRUS probe 17. For example, the system of the current invention (e.g., as illustrated in FIGS. 1-3) may be mounted on an existing and/or standard TRUS probe and/or TRUS probe mount (e.g., urological stepper) and/or TRUS probe template mount and/or brachytherapy platform.

According to some embodiments, an actuator may be provided for remote and/or manual and/or automatic translation of the cartridge 12 towards and/or away from template 13. Optionally, an actuator may drive cartridge 12 along a rail 16. Optionally, movement of cartridge 12 may be synchronized and/or independent of movement of TRUS probe 17. For example, as stylets 14 are drawn backwards during a treatment program, TRUS probe 17 may move with stylets 14 and/or as the treatment continues TRUS probe 17 may remain positioned in the same orientation with respect to stylets 14. Optionally, one or more sensors may be used to track the positioning of stylets 14 with respect to TRUS probe 17 and/or template 13.

According to some embodiments, the actuator may be connected to a controller. Optionally, the controller may be installed on the cartridge 12. Optionally, the controller may include individual on/off switches (relays) for some or all of the stylets. According to some embodiments, the controller may be automated, manual and/or remotely controlled.

According to some embodiments, the controller may be programmed to deliver the therapeutic effect as a result of focused ablation, electroporation, electrochemical or chemo-electroporation between local pairs of electrodes. Optionally, the controller may be programmed to deliver the therapeutic effect to the locations with high risk of disease. Alternatively, or additionally, the controller may be programmed to treat a whole gland or organ, for example, in each layer of the scan, all the stylets in the glad/organ may be activated. Optionally, the controller may be programmed to deliver the therapeutic effect interstitially at the location of the interstitial stylets.

According to some embodiments, the controller may be programmed to deliver the therapeutic effect selected from a group comprising: contact radiofrequency energy, non-contact radiofrequency energy, electroporation, ultrasonic energy, laser energy, gamma radiation, beta radiation, alpha radiation, immunotherapy, or a combination thereof.

According to some embodiments, the controller may be programmed to deliver current at a plurality of frequencies between various groups of said plurality of stylets.

According to some embodiments, the stylets may be introduced into the volume of tissue in mostly parallel directions using imaging modalities such as but not limited to: impedance, ultrasound (e.g., TRUS), computed tomography (CT), Magnetic resonance imaging (MRI), positron emission tomography (PET) scan, single-photon emission computerized tomography (SPECT) scan, fluoroscopy, endoscopy, laparoscopy, or any combination or fusion of modalities.

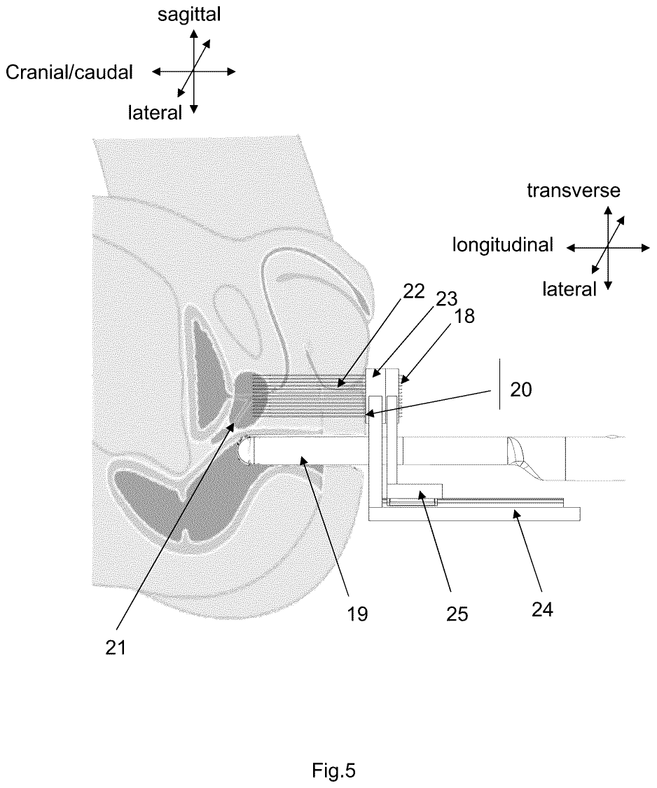

FIG. 5 is a schematic illustration of positioning of a system with an integrated TRUS probe in accordance with some embodiments of the current invention. For example, the system may be inserted multiple stylets 22 into the prostate 21 via the perineum 20. Optionally, actuator 25 may be provided for remote and/or manual and/or automatic translation of the cartridge 18 towards and/or away from template 23. Optionally, actuator 25 may drive cartridge 18 along a rail 24. Optionally, movement of cartridge 18 may be synchronized and/or independent of movement of TRUS probe 19. Optionally, the system may be integrated with a TRUS probe 19 which is inserted into the rectum. Optionally, stylets 22 may be positioned in the portions of the array that will intersect with the tissue of a procedure. Optionally, as the stylets 22 are moved through the tissue different arrays of stylets 22 may be activated in accordance with the position of areas of a procedure. Optionally the system may treat intersecting and/or non-intersecting volumes within a layer and/or across layers. In some embodiments, a system may be activated to map and/or treat multiple volumes simultaneously. Alternatively, or additionally, different areas may be mapped and/or treated sequentially.

In the embodiment of FIG. 5, the transverse, longitudinal and lateral directions of the device correspond to the sagittal, cranial caudal and lateral directions of the user's body respectively.

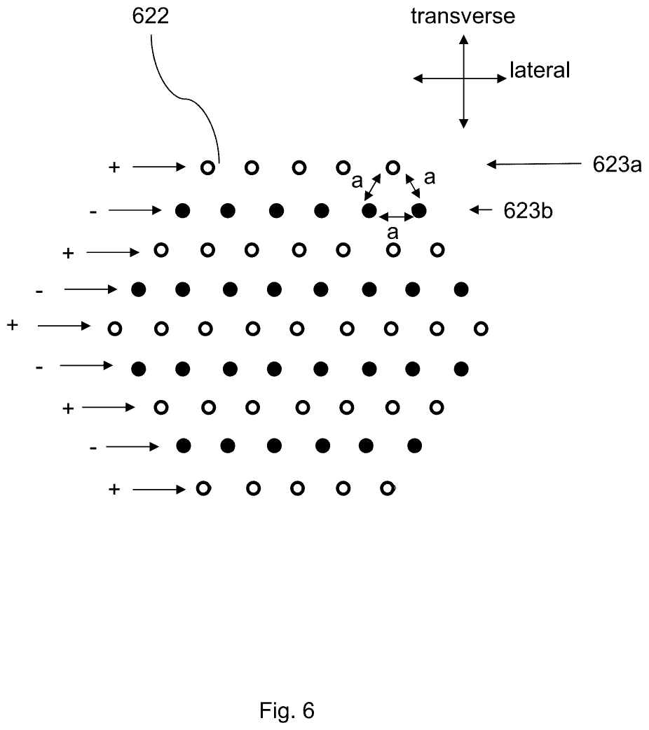

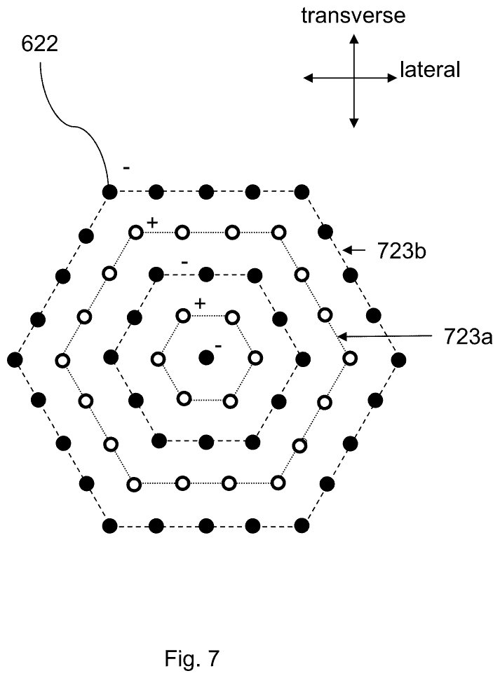

FIG. 6 is a front (axial) view of a schematic illustration of positioning and/or actuation of a plurality of stylets 622 in the transverse plane (defined as a plane perpendicular to the longitudinal axis and/or including the transverse and lateral axes. For example, in TRUS procedures the stylets may be nearly parallel (e.g., within 5 degrees and/or between 5 to 15 degrees and/or between 15 to 30 degrees) to the axial axis of the subject's body in accordance with some embodiments of the current invention. For example, hexagonal position of the stylets may ensure equal spacing (a) between consecutive stylets 622 both along lateral rows 623a, 623b and/or between rows. For example, stylets 622 in adjacent rows 623a, 623b may be off-set to preserve a desired distance. Optionally, adjacent rows 623a, 623b of stylets 622 will be activated with opposite polarities (for example, in FIGS. 6, 7 and 8 positive polarity stylus's 622 of row 623a are illustrated with a white filled circle and negative polarity stylus's 622 of row 623b) are illustrated with black circles). For example, for an alternating current, adjacent rows 623a, 623b may be synchronized to alternate simultaneously with opposite polarities. Alternatively, or additionally, polarity differences of the stylets 622 may be distributed in a different form, for example, alternating on adjacent columns in the transverse direction and/or in alternatively polarity along concentric rings (e.g., as illustrated in FIG. 7) etc. Alternatively, or additionally, stylets may not be distributed evenly. Alternatively, or additionally, differences in polarity and/or activation may be distributed differently in the matrix. Alternatively, or additionally, there may be an external electrode and/or all of the stylets may have the same polarity. In some embodiments, the geometry of differing polarities may be changed over time e.g., to achieve more even treatment. Alternatively or additonally, stylets of opposing polarity may be distanced at a constant gap while stylets of similar polarity may be distributed in a different gap and/or in varying gaps.

Multiple stylets may be connected simultaneously to positive and negative polarities of the electrical source. For example, in FIG. 6, each consecutive row is connected to a different polarity.

FIG. 7 is a front (axial) view of a schematic illustration of positioning of a plurality of stylets 622 in accordance with some embodiments of the current invention. In some embodiments, polarity activation of stylets may be arranged in concentric patterns (e.g., hexagonal rings 723a, 723b) where adjacent patterns have opposite polarities. For example, an outer hexagonal ring 723b of stylets may have a negative polarity while an inner hexagonal ring 723a of stylets 622 may have a positive polarity. Optionally, the patterns may be closed forms (e.g., hexagons, rectangles, irregular shapes etc.) alternatively or additionally, the patterns may be open forms (e.g., open ended rectangles, incomplete triangles etc.)

For example, in FIG. 7, each consecutive hexagonal rings 723a, 723b (depicted by dashed and dotted lines) is centered on the center of the stylet array is connected to a different polarity. Alternatively, or additionally, such arrays of concentric sets of stylets with opposing pluralities may not be centered on the center of the array and/or multiple areas of concentric sets of opposite charged stylets may be activated in the array of stylets.

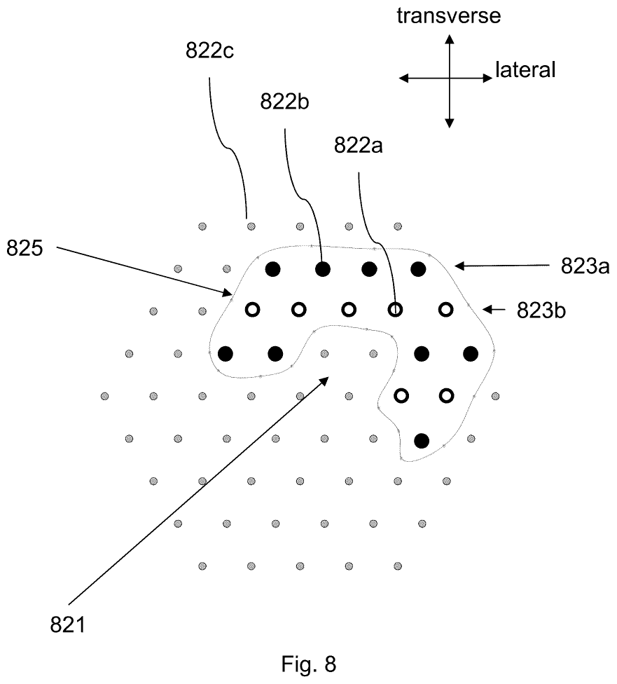

FIG. 8 is an axial cross-sectional schematic illustration of positioning of a plurality of stylets 822a, 822b, 822c wherein an ablation area is selected, in accordance with some embodiments of the current invention. For example, a region of a procedure 824 is selected (e.g., via a graphic interface). Optionally, all the stylets 822a outside of the selected contour may be turned off and/or disconnected from current. Optionally, stylets 822a, 822b in the selected contours are activated, for example, in rows 823a, 823b wherein adjacent 822a, 822b rows have opposing polarity. In the displayed embodiment there is one area of a procedure 824. Optionally there may be multiple areas of treatment within one plane. Optionally, the area of treatment may change from plane to plane as the set of stylets moves longitudinally. Optionally, the pattern of polarities may be selected to achieve good coverage of the region of a procedure 824 and/or avoid affecting healthy tissue. For example, in a non-convex region of a procedure 824, the pattern of polarities may be selected to avoid current passing through healthy tissue which protrudes into indentations 821 in the region of a procedure 824 (e.g., nearby stylets 822a, 822b in the region of a procedure 824 with a portion of healthy tissue intervening between them may be selected to have the same polarity).

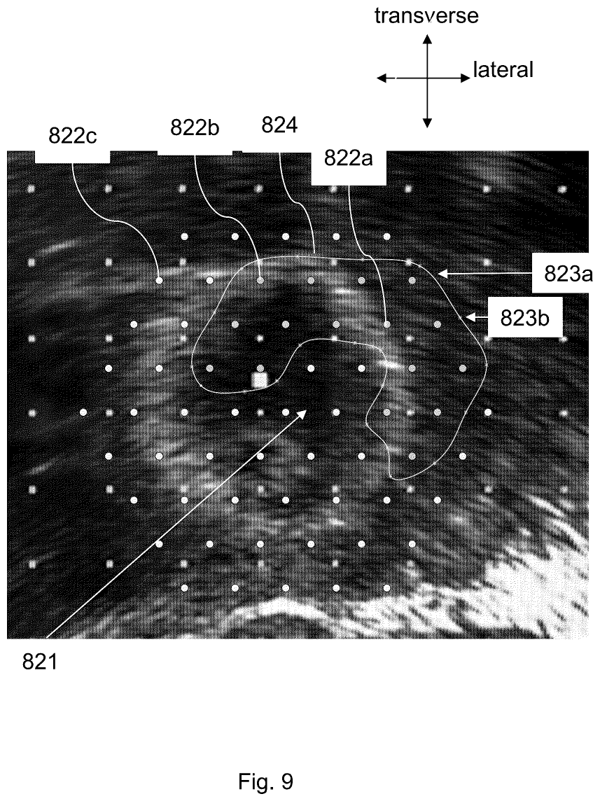

In some embodiments, the graphic interface may include a graphical display including data from one or more imaging processes, such as ultrasound, MRI, CT, X-ray, impedance tomography, etc. (for example, as illustrated in FIG. 9). Optionally, data from one or more imaging processes may be merged. Optionally, one or more stylets may be observed on the graphical display. Optionally, one or more stylets may be selected from the graphical display. Optionally, one or more stylets may be activated from the graphical display and/or selected for activation, e.g., the polarity, radiofrequency, duty cycle, strength, voltage, current, etc.

FIG. 9 is an exemplary ultrasound image of a plurality of stylets wherein an area of ablation is selected in accordance with some embodiments of the current invention.

In this particular embodiment, stylet position may be fused with ultrasound imaging. An area for ablation is selected via an operator interface (e.g., a graphic user interface).

In some embodiments, the operator interface is configured for the operator marking on the real time image a region of a procedure. For example, overlay may include a diagnostic image. The operator may recognize the diagnostic image an area of diseased tissue and/or mark the region for a procedure 824 and/or a diagnostic procedure. Alternatively or additionally, the overlay may include a real time image (e.g., of impedance measurements) that shows a progress of a procedure. The operator may recognize and/or mark an area for further treatment.

In some embodiments, the marking is performed at various depths. For example, the area of a procedure may differ from layer to layer through the tissue. Optionally, before performing a procedure on a first layer an operator may mark are region in that layer to be acted upon. The stylets may be moved (for example the stylets may be retracted together to another layer of the tissue). Optionally, in the second layer the operator may mark a different region and the procedure in the second layer may be operated upon in the region marked in that layer.

In some embodiments, the system further includes a processor running an algorithm to activate only those stylets 822a, 822b in the region of a procedure at each of the various depths. Optionally, an electronic grid is superimposed on the real time image. For example, the grid markings may coincide with expected positions of the stylets after being pushed into tissue.

In some embodiments, the system includes a processor running an algorithm to activate a remote actuator (e.g., a pusher). Optionally, the pusher pushes selected stylets into the tissue at the desired depth and within the boundaries of the region of a procedure. In some embodiments, the processor runs an algorithm to pass current from a first subset of the stylets (e.g., row 823a) to a second subset of the stylets (e.g., row 823b) that are intermingled with each other. Optionally, stylets 822c outside of the selected contour and/or region of the procedure 824 may be turned off and/or disconnected.

In some embodiments, the pattern of stylets may be rotatable around a longitudinal axis. For example, this may be used to align the rows of the stylets with a long axis of a region of a procedure.

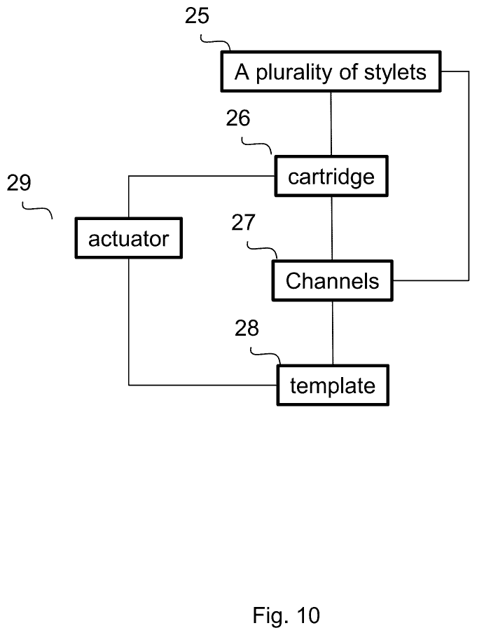

FIG. 10 is a block diagram of evaluation of an electroporation system in accordance with an embodiment of the current invention. For example, a plurality of stylets 22 connected to cartridge 26 may pass through tight channels 27 in template 28, wherein cartridge 26 is slidable with parallel guidance rods and/or linear bearing and/or bushing 29. Optionally, the multiple stylet system of the current invention may be configured for mounting on a TRUS probe stepper system. For example, the multiple stylet system may be mounted to a mount for a template. Optionally, an actuator may move the set of stylets synchronously with and/or independently of the TRUS probe. For example, an actuator (e.g., a linear actuator, a DC motor, a stepper motor, etc.) may move the cartridge 26 with respect to the template. The actuator may be controlled by an operator, e.g., using a controlled power source and/or a controller.

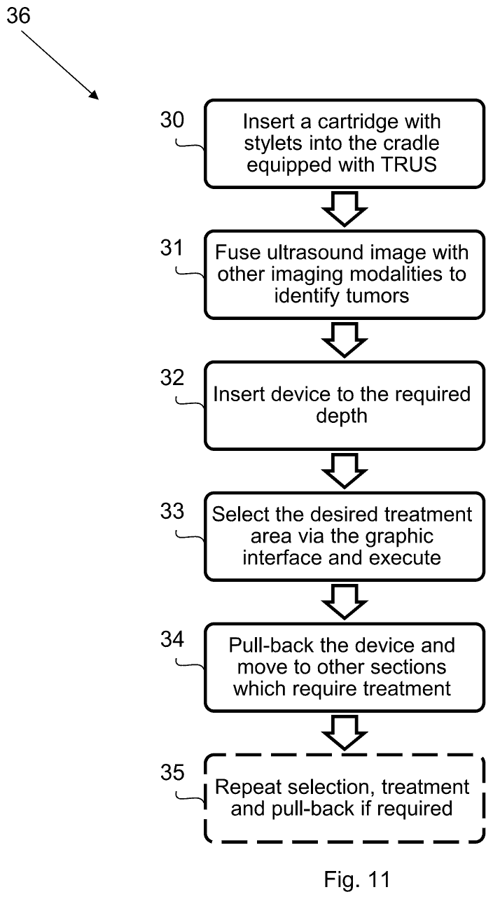

FIG. 11 is a flow chart of treatment of a tumor using an electroporation system in accordance with an embodiment of the current invention. For example, in method 36, a cartridge with an array of stylets may be inserted 30 into a cradle (e.g., of a urological stepper), equipped with transrectal ultrasound (TRUS). Fuse 31 the ultrasound image with one or more other imaging modalities to identify one or more tumors in the tissue (e.g., prostate). The cartridge may be connected to a set of stylets. For example, a proximal portion of the stylets may be connected to the cartridge in a two-dimensional array (e.g., arrayed over the lateral and/or transverse directions. Optionally, the stylets are oriented with their longitudinal axis perpendicular to the cartridge (lateral and/or transverse directions). Optionally, the cartridge is configured to move with the proximal portion of the stylets in the longitudinal direction. For example, all of the stylets may move in a coordinated manner with the cartridge. Optionally, the proximal portion of the stylets may be locked to the cartridge.

In some embodiments, the system may include a quick electrical connector and/or a multi-channel electrical connector to connect various channels electrically to the various stylets. For example, the cartridge may include this connector. For example, by powering one or more of the channels the cartridge may extend and/or activate some or each stylet. For example, a stylet my include an electrode on a proximal end or proximal portion thereof and/or each electrode may be activated by an operator of the system. Alternatively or additionally, each stylet may have an address code and/or may be controlled (e.g., activated, deactivated, extended and/or retracted individually) by a controller.

Optionally, the system may include a quick connector to physically lock the stylets to the existing infrastructure. Optionally, the system may be configured to facilitate easy positioning of the cartridge. Optionally, the stylet cartridge and the electrical connectors may permit easy introduction and removal. Optionally, the cartridge, stylets and/or template may be single use and/or disposable. Alternatively, or additionally, the cartridge, stylets and/or template may be reusable and/or sterilizable. In some embodiments, the cartridge, stylets and/or template may be supplied as a kit. Alternatively or additionally, the cartridge, stylets and/or template may be supplied individually. Additionally or alternatively, the cartridge may include a stopper preventing disengagement.

In some embodiments, the array stylets may be inserted 32 through the perineum to the required depth, e.g., a maximal required depth. Optionally, insertion may be accomplished manually, automatically, robotically and/or remotely. The required region of a procedure is selected 33. Optionally, treatment may be executed in the selected area. Optionally, the system may be moved longitudinally, e.g., pulled-back (e.g., proximally) 34 and/or translated forward (e.g., distally), e.g., stylets are retracted in the longitudinal direction the system stops every few millimeters and treats a new layer in the transverse and lateral directions. Optionally, the system is then pulled back 34 and moved to other sections (e.g., depths, locations, etc.) which require treatment. This may be accomplished manually, automatically, robotically and/or remotely. Optionally, repeat the previous steps may be repeated 35 as many times as required.

In some embodiments, the region of a procedure may be selected manually, e.g., via a graphical interface of the fused image modalities. Alternatively, or additionally, selection may be automatic with some algorithmics, such as AI, Machine Learning and/or signal processing. Optionally, the region of a procedure may be pre-designated.

In some embodiments, the system may pull the stylets through the tissue and/or activate them according to the pre-designating. Pre-designation may shorten the treatment process. Optionally, the system may include some integration of automatic and/or manual control. For example, during automatic treatment, the system may wait for manual authorization by an operator at various steps in the treatment process. The treatment may be selected from: irreversible electroporation (IRE), reversible electroporation (RE), instillation of chemotherapeutics, calcium solution or gene therapy, Radiofrequency ablation, microwave ablation, laser ablation, photodynamic therapy and/or a combination thereof.

In some embodiments, the therapeutic effect may be selected from a group including: contact radiofrequency energy, non-contact radiofrequency energy, electroporation, ultrasonic energy, laser energy, gamma radiation, beta radiation, alpha radiation, immunotherapy, or a combination thereof. Optionally, electrical ablation may induce electroporation. Optionally, electroporation may be delivered in pulses. Optionally, electroporation may be used in conjunction with one or more chemotherapeutic compounds. Optionally, the electroporation used in conjunction with one or more chemotherapeutic compounds may be synergistic. Optionally, the chemotherapeutic may be an anti-cancer compound. Optionally, the anti-cancer compound may be a small molecule, a biological molecule such as an antibody, a metal, an organometallic compound, and/or a radio isotope.

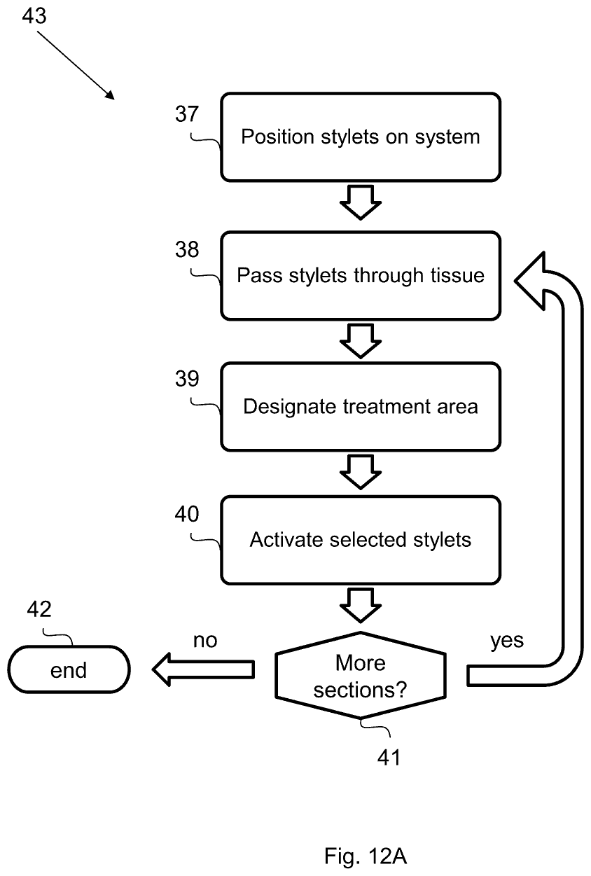

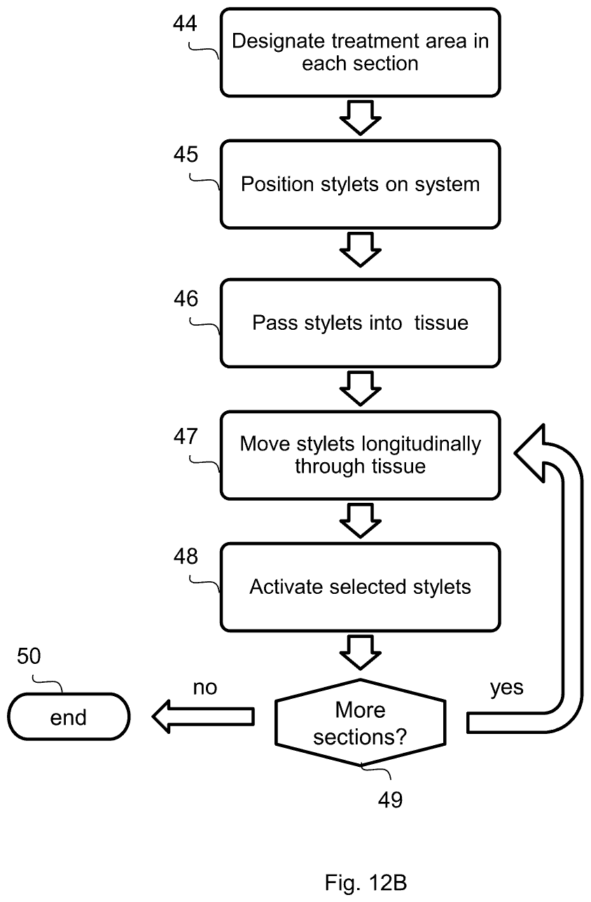

FIGS. 12A and 12B are flow charts of treatment and/or mapping of tissue in accordance with embodiments of the current invention. For example, in method 43, an array of stylets may be mounted 37 to a system for insertion and/or activation of stylets. Optionally, the stylets are passed 38 into a target tissue. Optionally, the stylets may be passed into the target tissue, sequentially, in groups, and/or simultaneously. Optionally, a region of a procedure is designated 39. Selected stylets and/or electrodes are inserted into the region of a procedure and activated 40. Additionally, or alternatively, an electrode outside the region of a procedure may be activated (for example a general ground and/or return electrode). Optionally, the stylets may be moved longitudinally 47 from layer to layer and/or treatment and/or sensing is performed in another layer of the tissue 41, if required. Optionally, if no further treatment and/or monitoring is required, the treatment may be ended 42. Optionally, if further treatment and/or monitoring is required, one or more of the previous steps may be repeated.

In some embodiments, the region of a procedure of each section/layer may be designated 44. Optionally, an array of stylets may be mounted 45 to a system for insertion and/or activation of stylets. Optionally, the stylets are passed 46 into a target tissue to the designated area. Optionally, the stylets may be passed into the target tissue, sequentially, in groups, and/or simultaneously. Selected stylets and/or electrodes are inserted into the region of a procedure and activated. Additionally, or alternatively, an electrode outside the region of a procedure may be activated (for example a general ground and/or return electrode). Optionally, the stylets may be moved 47 longitudinally (e.g., pulled back or translated forward) from layer to layer and/or treatment and/or sensing is performed in each layer of the tissue 49, after moving the electrodes to that layer (e.g., based on the data from the treatment and exploration), for example as illustrated in FIG. 12A, if required. Optionally, selected stylets may be activated 48. Optionally, the selected stylets may be activated sequentially, in groups, and/or simultaneously. Optionally, if no further treatment and/or monitoring is required, the treatment may be ended 50. Optionally, if further treatment and/or monitoring is required, one or more of the previous steps may be repeated.

Alternatively, or additionally, there may be a preliminary designation step (e.g., with the TRUS probe without stylets and/or via another sensing technology [e.g., biopsy, tomography (e.g., MRI, CAT etc.)]. Based on the designation step, for example as illustrated in FIG. 12B. In such a case the treatment steps and movement 47 of the stylets through the tissue may be rapid.

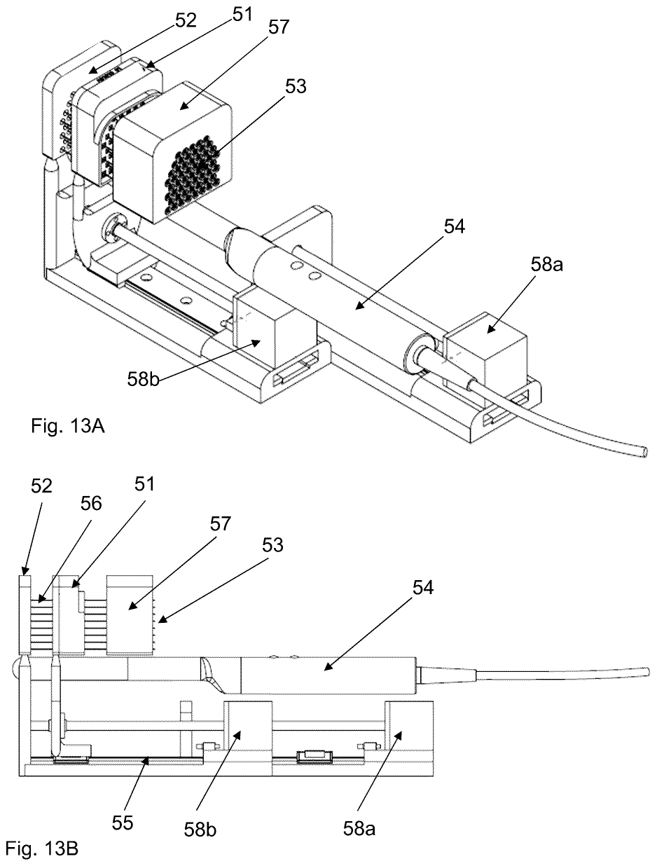

FIGS. 13A and 13B are a perspective view and a side view, respectively, of schematic illustrations of positioning of a system with an integrated TRUS probe, in accordance with some embodiments of the current invention. For example, the system may include a front face with a first template 52. Optionally, the template may facilitate correct spacing and/or positioning of the stylets. For example, the first template 52 may include holes through which stylets 56 are inserted into tissue. For example, the first template 52 may be placed against the skin of a subject (e.g., a perineum) guiding the stylets 56 to the intended tissue.

According to some embodiments, each of the stylets and/or cartridge may include stylet indicators 53. Optionally, the stylet indicator 53 may be a marking on the end and/or side of the stylet (e.g., numbers and/or LED's). Optionally, the stylet indicator 53 may be configured so that the user can see which stylets are extended and/or autoactivated (e.g., the electrode is actively receiving current) at any time. Optionally, the stylets, template, cartridge and/or indicators may be packaged together and/or single use. Optionally, the system may be mounted on an existing brachytherapy platform e.g., alongside a TRUS probe 54.

According to some embodiments, each stylet may be extended and/or retracted individually. Optionally, individual movement may facilitate changing the geometry of the array of active elements for each layer for a 3D tumor and/or for a patient based on the geometry of the tissue needing to be treated and/or explored. Optionally, the stylets 56 may be moved together, e.g., the stylets may be connected to a cartridge, and/or template, and/or template. Optionally, the stylets may be moved together. For example, the stylets may be inserted together and then retracted individually and/or in groups to facilitate changing the geometry of the array of active elements for each layer to accommodate the unique geometry of each tumor. Optionally, the stylets may be extended and/or retracted manually, automatically and/or remotely.

In some embodiments, the system in include a second template 51. For example, the second template 51 may be positioned between the first template 52 and the cartridge 57. The second template 51 is optionally connected to at least a portion of the stylets 56. Optionally, an actuator 58b moves the second template 51. For example, moving the second template 51 longitudinally by the actuator 58b with respect to the first template 52 may move the portion of the stylets (and/or all of the stylets 56 and/or the cartridge 57) longitudinally (e.g., inserting and/or retracting the stylets 56). In some embodiments, the second template 51 may move stylets 56 independently of the cartridge 57. Alternatively or additionally, the cartridge 57 may move with some or all of the stylets 56.

In some embodiments, the second template 51 may be configured for retracting the stylets for a predetermined distance. Optionally, a processor running is configured for the retracting and/or for positioning active electrodes to a prescribed depth. For example, after treating a layer at a first depth, the second template 51 will be moved back longitudinally (e.g., away from the first template 52). Optionally, this moves the stylets 56 and/or electrodes to a next more superficial depth (e.g., layer) of a procedure. In some embodiments, the retracting and/or the distance between layers ranges between ½ cm to 3 cm. As a selected set of the stylets 56 and/or electrodes pass from layer to the layer through the tissue, different subsets of the moving stylets 56 and/or electrodes may be activated. For example, in a layer where the region of the procedures is large, a large number of stylets 56 (e.g., all of the inserted stylets 56) may be activated. In another layer wherein the region of the procedure is smaller, only a portion of the inserted stylets 56 may be activated. Optionally, a processor will control which stylets 56 are inserted and/or which stylets and/or electrodes are activated in which layers of the tissue.

In some embodiments, one or more of the templates 51, 52 may be rotated around a longitudinal axis. For example, this may facilitate orienting the stylets 56 with a region of a procedure. For example, the rows of the stylets 56 may be aligned with a long axis of the area of the procedure.

According to some embodiments, the system may facilitate insertion of multiple transperineal stylets 56 together. Optionally, the system may facilitate controlled insertion of a 2D array of transperineal stylets. Optionally, the system may facilitate positioning of the transperineal stylets and a TRUS imaging probe. Optionally, the system may facilitate synchronization of positioning of the transperineal stylets and a TRUS imaging probe. Optionally, one or more stepper motors may insert and/or retract the transperineal stylets and a TRUS imaging probe. Optionally, the one or more stepper motors 58b of the stylets may function independent of the TRUS stepper motor 58a. Optionally, the stepper motor may facilitate axial advancement of the TRUS probe. Optionally, the stepper motor may facilitate rotation of the TRUS probe for imaging the stylet advancement in the longitudinal plane. Optionally, the angle of rotation is determined automatically by the predetermined position of the stylet position in the cartridge relative to the position the TRUS as resulting from the geometry of the cartridge and stepper design.

According to some embodiments, the one or more stepper motors of the stylets may be configured to function in synchronization with the TRUS stepper motor. Optionally, a stepper motor may be configured for movement of stylets 56, and/or a first template 52, and/or front plate, and/or cartridge 57. Optionally, the stepper motors may move along a rail 55. Optionally, the stylets may be extended and/or retracted in the longitudinal direction by one or more stepper motors such that the system may stop every few millimeters to treat a new tissue layer in the transverse and lateral directions.

According to some embodiments, the system and TRUS may be held in pre-determined positions reducing the mutual degrees of freedom and/or in relation to the patient by a stepper system connected to the patient bed and/or patient.

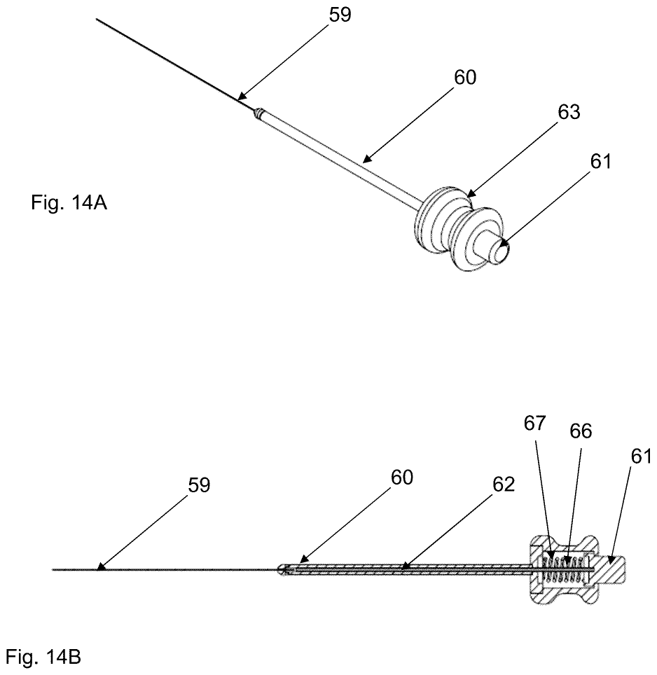

FIGS. 14A and 14B are a perspective view and a side view, respectively, of schematic illustrations of an exemplary transperineal stylet, in accordance with some embodiments of the current invention. For example, each stylet may comprise a telescopic, removable, and/or retractable inner rod 59. Optionally, each stylet may be at least partially covered by a sheath 60. Optionally, sheath 60 may be electrically insulating, thermally insulating, or both. Optionally, sheath 60 may be composed of a polyimide tube. Optionally, sheath 60 may be removable and/or retractable. Optionally, each of the stylets may include stylet indicators 61. Optionally, stylet indicator 61 may be a marking on the end and/or side of the stylet (e.g., numbers and/or LED's). Optionally, the stylet indicator 61 may be configured so that the user can see which stylets are extended and/or activate (e.g., the electrode is actively receiving current) at any time. Optionally, each stylet may include a lumen. Optionally, the lumen may be configured to contain one or more electrodes 62.

In some embodiments, some or all of the stylets 681 may include a pusher for moving the inner rod 59 longitudinally. For example, the pusher may comprise a magnetic tip on a proximal portion 66 of the rod 59 that interfaces with a magnetic or magnetizable means (e.g., a coil 67) on the tail 63 of the stylet for releasably engaging the each of the rods 59. Optionally, the pusher, is provided with a slidable actuator for detaching the pusher from the engaged rod 59.

Optionally, each stylet may be extended and/or retracted individually. Optionally, each stylet may be housed in a cartridge. Optionally, the cartridge may include a magnetic tip. Optionally, the magnetic tip may interface with a magnetic and/or magnetizable means on the tail 63 of the stylet for releasably engaging the stylet. Optionally, the system may include a slidable actuator for detaching from the engaged stylet. Optionally, individual stylet movement may facilitate changing the geometry of the array of active elements for each layer for a 3D tumor. Optionally, each stylet may be introduced to the system one by one according to a predetermined plan under imaging guidance. Optionally, such insertion may be controlled manually, automatically and/or remotely.

In some embodiments, the system includes an x-y actuator that positions a pusher against a selected stylet and/or group of stylets. The pusher is then activated to insert and/or retract the selected stylets. For example, the pusher may be connected to an actuator for pushing pushes the selected stylets to a desired depth. Optionally, the system also includes a second actuator for detaching the pusher from the selected stylet.

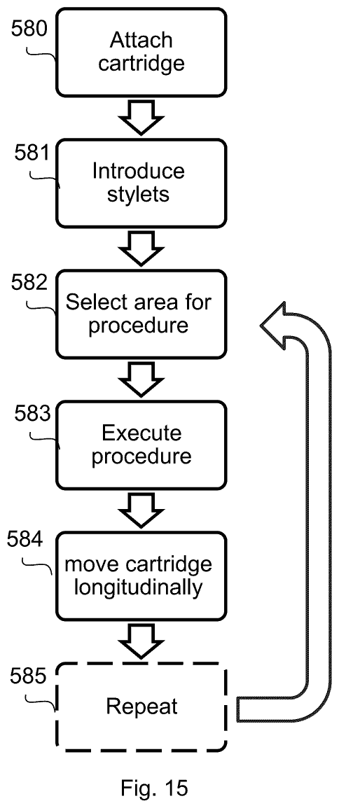

FIG. 15 is a flow chart illustration of a method of executing a procedure in accordance with an embodiment of the current invention. Optionally, the procedure may include a treatment for a condition (e.g., ablation of diseased tissue) and/or an exploratory procedure (e.g., a biopsy and/or an imaging (for example, electro impedance imaging)).

In some embodiments, a cartridge with an array of stylets is attached 580 to a cradle of an imaging modality. Optionally, the cartridge may be configured for parallel and/or synchronized introduction 581 of stylets into the tissue. For example, the stylets may be introduced 581 transperineally into the tissue to a desired depth. Optionally, a desired region of a procedure may be selected 582. In some embodiments the region of a procedure may be selected under monitoring by the imaging modality. Alternatively or additionally, the region of a procedure may have been preselected.

In some embodiment, the procedure (e.g., treatment and/or exploratory procedure) may be executed 583 on the selected 582 region of a procedure. After treatment of one layer of the region of a procedure, the stylets may be moved 584 longitudinally (e.g., further inserted and/or retracted) to another layer and/or the previous steps may be repeated 585 (e.g., until the procedure is finished and/or until the entire region of a procedure is covered).

In embodiments, the imaging modality may include ultrasound, computed tomography (CT), magnetic resonance imaging (MRI), positron emission tomography (PET) scan, single-photon emission computerized tomography (SPECT) scan, fluoroscopy, endoscopy, laparoscopy, or any combination thereof. Alternatively, or additionally, the imaging modality may include Trans Rectal Ultrasound (TRUS).

In some embodiments, introducing 581 and/or retracting of the stylets is accomplished manually, automatically, robotically, remotely or any combination thereof. Optionally, the stylets are retracted in a longitudinal direction and/or the retracting is stopped periodically for treating a new layer in the transverse and lateral directions.

In some embodiments, the region of a procedure is selected manually or automatically. Alternatively or additionally, the region of a procedure may be pre-designated.

In some embodiments of the invention, the procedure includes contact radiofrequency energy, non-contact radiofrequency energy, electroporation, ultrasonic energy, laser energy, gamma radiation, beta radiation, alpha radiation, immunotherapy, or a combination thereof.

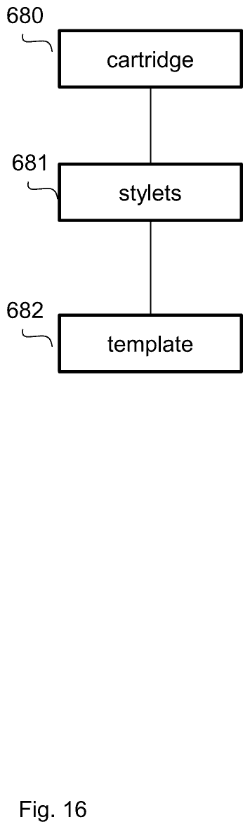

FIG. 16 is a block diagram illustrating a system for inserting stylets into tissue in accordance with an embodiment of the current invention. In some embodiments, a cartridge 680 houses multiple stylets 681. Optionally, a template 682 directs the stylets 681 into tissue. For example, the template 682 may include channels through which a distal portion of the stylets pass into the tissue. For example, the template 682 may be positioned distal to the cartridge 680 and/or the cartridge 680 may be configured to move longitudinally with respect to the template 682. In some embodiments, the stylets are parallel and/or oriented longitudinally.

In some embodiments, the tissue into which the stylets 681 are inserted includes a prostate. For example, the stylets may be introduced through the perineum.

In some embodiments, the includes an actuator for pushing the stylets into tissue. Optionally, the system includes an X-Y actuator for moving a pushing mechanism opposite the tail of a stylets 681 housed within the cartridge 680. Optionally, the actuator pushes the cartridge 680 and/or the stylets to a desired depth into the tissue.

In some embodiments, introduction of the stylets 681 is performed under imaging guidance. For example, the imaging may include CT, MRI, PET, fluoroscopy, laparoscopy, endoscopy. For example, the imaging may include transrectal ultrasound TRUS. Optionally, the patient is in gynecologic position and/or with legs in stirrups. In some embodiments, the TRUS probe is held in a predetermined relation to the stylets 681, the cartridge 680 and/or the template 682. For example, this may reduce the mutual degrees of freedom and/or in relation to the patient. Optionally, movement of the TRUS, the stylets 681, the cartridge 680 and/or the template 682 is by a by a stepper system connected to the patient bed and/or the patient. For example, the actuator causes for axial advancement of the TRUS, the stylets 681, the cartridge 680 and/or the template 682. For example, the advancement may be synchronized and/or independent.

In some embodiments, there may be another actuator for rotation the TRUS for imaging the needle advancement in the longitudinal plane. Optionally, the angle of rotation is determined automatically by the predetermined position of the needle position in the cartridge 680 relative to the position the TRUS as resulting from the geometry of the cartridge 680 and stepper hardware.

In some embodiments, the stylets 681 are introduced automatically according to a predetermined plan and/or under imaging guidance.

Optionally, some or all of the stylets 681 may include a pusher for moving the individual stylet longitudinally. For example, the pusher may comprise a magnetic tip that interfaces with a magnetic or magnetizable means on the tail of the stylets 681 for releasably engaging the each of the stylets 681. Optionally, the pusher, is provided with a slidable actuator for detaching the pusher from the engaged needle.

In some embodiments, the system includes visual marks for the position of the stylets 681. The markings may indicate which stylet is which on the cartridge 680 and/or which of stylets 681 has been introduced into the tissue according to a marking on physician screen interface. For example, the visual marking may include LED marking, image projection by a dedicated adjacent projecting means.

These embodiments are provided by way of example and are in no means intended to limit the scope of the invention.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

### General

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

The term “impedance” as used herein refers to the opposition to alternating current presented by the combined effect of resistance and reactance in a circuit.

The term “impedance tomography” as used herein refers to a noninvasive type of medical imaging in which the electrical conductivity, permittivity, and impedance of a part of the body is inferred from surface electrode measurements and used to form a tomographic image of that part.

The term “spectroscopy” as used herein refers measurement and interpretation of the electromagnetic spectra that result from the interaction between electromagnetic radiation and matter as a function of the wavelength or frequency of the radiation.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are Optionally, provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Data and/or program code may be accessed and/or shared over a network, for example the Internet. For example, data may be shared and/or accessed using a social network. A processor may include remote processing capabilities for example available over a network (e.g., the Internet). For example, resources may be accessed via cloud computing. The term “cloud computing” refers to the use of computational resources that are available remotely over a public network, such as the internet, and that may be provided for example at a low cost and/or on an hourly basis. Any virtual or physical computer that is in electronic communication with such a public network could potentially be available as a computational resource. To provide computational resources via the cloud network on a secure basis, computers that access the cloud network may employ standard security encryption protocols such as SSL and PGP, which are well known in the industry.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A system for insertion of an array of stylets into tissue of a subject, comprising:
a cartridge housing a plurality of stylets each of said stylets including a pointed distal end and a proximal portion connected to the cartridge; and
a first template with a plurality of channels through which a distal portion of the stylets pass into the tissue.

2. The system of claim 1, further comprising an actuator configured to move said stylets longitudinally with respect to said first template thereby moving said distal portion of said plurality of stylets longitudinally through said channels of said first template.

3. The system according to claim 2, further comprising an x-y actuator for positioning a pusher against a selected stylet of said plurality of stylets and wherein said actuator pushes the selected stylet to a desired depth.

4. The system according to claim 1, further comprising a second template, said first template in contact with the subject and the second template positioned between the first template and the cartridge, wherein the second template is connected to at least a portion of said stylets and wherein said second template is moved longitudinally by an actuator between said first template thereby moving said portion of the stylets longitudinally.

5. The system of claim 4 wherein said second template is configured for retracting said stylets for a predetermined distance.

6. The system according to claim 5, further comprising a processor running an algorithm configured for said retracting and for positioning active electrodes to a next more superficial depth of a procedure.

7. The system according to claim 1, comprising an operator interface providing a real time image of the tissue, wherein the operator interface is configured for the operator marking on the real time image a region of a procedure to perform a procedure in two disconnected regions simultaneously.

8. The system according to claim 7, wherein the marking is performed at various depths, further comprising a processor running an algorithm to activate to activate only stylets of said plurality of stylets in the region of a procedure at each of said various depths.

9. The system according to claim 1, further comprising a bioimpedance module that is connected to the stylets for testing impedance between electrodes on the stylets.

10. The system of claim 1, wherein the at least one of the cartridge and the stylet includes an individual actuator for longitudinal movement of an individual stylet.

11. The system according to claim 1, wherein each stylet includes a lumen and wherein the lumen is configured to permit insertion of active electrodes.

12. The system according to claim 1, wherein each stylet is at least partially covered by a covering including at least one of a sheath and a coating.

13. The system according to claim 12, wherein the sheath is removable or retractable.

14. The system according to claim 1, wherein each of said stylets is provided with at least two sensing electrodes for bioimpedance measurements.

15. The system according to claim 1, wherein the template is configured to provide a variety of densities and patterns of the plurality of stylets.

16. A method for electro-stimulation of tissue of a subject, the method comprising:
attaching a cartridge with an array of stylets to a cradle of an imaging modality, wherein the cartridge is configured for parallel insertion of stylets into the tissue;
introducing the stylets into the tissue to a desired depth, under monitoring by the imaging modality;
selecting a desired area;
executing a procedure on the selected area;
retracting the stylets to a more superficial transverse plane and repeating the previous steps, as needed.

17. The method according to claim 16, wherein the retracting is in a longitudinal direction and wherein retracting stops periodically for a procedure on a new layer in the transverse and lateral directions.

18. The method according to claim 16, wherein the area is pre-designated.

19. The method according to claim 16, wherein the procedure is selected from a group including: contact radiofrequency energy, non-contact radiofrequency energy, electroporation, ultrasonic energy, laser energy, gamma radiation, beta radiation, alpha radiation, immunotherapy, or a combination thereof.

20. The method according to claim 16, wherein multiple stylets are configured for acting simultaneously.
